(19) United States
(12) Patent Application Publication
(10) Pub. No.: US 2025/0098977 A1
(43) Pub. Date: Mar. 27, 2025

(54) Imaging Apparatus for Magnetic Resonance Device

(71) Applicant: Siemens Healthineers AG, Forchheim (DE)

(72) Inventor: Shuang Ji, Shenzhen (CN)

(73) Assignee: Siemens Healthineers AG, Forchheim (DE)

(21) Appl. No.: 18/888,444

(22) Filed: Sep. 18, 2024

(30) Foreign Application Priority Data
Sep. 22, 2023 (CN) 202322601058.9

(51) Int. Cl. A61B 5/055

(52) U.S. Cl. CPC A61B 5/055

(57) ABSTRACT

An imaging apparatus for an MR device, including: an image broadcast apparatus located in a first space; an optical/electrical conversion module, which is connected to the image broadcast apparatus using an optical fiber, and converts an image broadcast by the image broadcast apparatus into electrical signals; an electrical/optical conversion module, which is connected to the optical/electrical conversion module, and converts the electrical signals into the image; and a projection device, which is connected to the electrical/optical conversion module using an image transmission assembly, and projects the image to a top wall of a magnetic resonance chamber. The image transmission assembly has an image transmission lens group and/or an image transmission optical fiber bundle. The MR device, the optical/electrical conversion module, the electrical/optical conversion module, and the projection device are located in a second space. The first space and the second space are electromagnetically shielded from each other.

### TECHNICAL FIELD

This disclosure relates to magnetic resonance examination devices, in particular, an imaging apparatus for a magnetic resonance device.

### BACKGROUND

Magnetic resonance imaging is an important clinical imaging tool. Due to the presence of a strong magnetic field, it is difficult to add electronic indication signals inside magnets of existing nuclear magnetic devices, with the result that a patient does not know the progress of the magnetic scan and must wait for a diagnosis to finish while lying quietly inside a narrow magnet. Moreover, since the space in most existing magnetic resonance imaging devices is narrow and sealed, the patient easily becomes tense and anxious during the waiting process, affecting imaging quality.

### SUMMARY

In view of the defects of the prior art mentioned above, an object of the present disclosure is to provide an imaging apparatus for a magnetic resonance device to mitigate the problem that it is difficult to add electronic indication signals inside magnets of existing nuclear magnetic devices, such that a patient must wait for a diagnosis to finish while lying inside a magnet, yet since the space in most existing magnetic resonance imaging devices is narrow and sealed, the patient easily becomes tense and anxious during the waiting process, thus affecting imaging quality.

To achieve the above object and other related objects, the present disclosure proposes an imaging apparatus for a magnetic resonance device, the magnetic resonance device comprising an examination table and a magnetic resonance chamber, and the imaging apparatus comprising:
an image broadcast apparatus, which is located in a first space;
an optical/electrical conversion module, which is connected to the image broadcast apparatus by means of an optical fiber, and converts an image broadcast by the image broadcast apparatus into electrical signals;
an electrical/optical conversion module, which is connected to the optical/electrical conversion module, and converts the electrical signals into the image; and
a projection device, which is connected to the electrical/optical conversion module by means of an image transmission assembly, and is used for projecting the image to a top wall of the magnetic resonance chamber,
wherein the image transmission assembly at least comprises an image transmission lens group and/or an image transmission optical fiber bundle; and the magnetic resonance device, the optical/electrical conversion module, the electrical/optical conversion module, and the projection device are located in a second space, and the first space and the second space are electromagnetically shielded from each other.

In one aspect of the present disclosure, the electrical/optical conversion module is a DLP optical engine.

In one aspect of the present disclosure, the projection device is arranged at one end of a table body of the examination table that is near the magnetic resonance chamber.

In one aspect of the present disclosure, the imaging apparatus further comprises an electromagnetic shielding box, which is mounted inside a base of the examination table, the optical/electrical conversion module and the electrical/optical conversion module being arranged inside the electromagnetic shielding box.

In one aspect of the present disclosure, the image transmission lens group is mounted inside a base of the examination table or on a table body of the examination table.

In one aspect of the present disclosure, the image transmission assembly comprises an image transmission lens group and an image transmission optical fiber bundle, and two ends of the image transmission optical fiber bundle are respectively connected to the image transmission lens group and the projection device.

In one aspect of the present disclosure, the projection device comprises a support and an image projection lens group, the support being mounted at one end of a table body of the examination table that is near the magnetic resonance chamber, and the image projection lens group being fixed on the support.

In one aspect of the present disclosure, the support comprises an optical fiber bundle fixing structure, and one end of the image transmission optical fiber bundle that is remote from the image transmission lens group is fixed on the optical fiber bundle fixing structure.

In one aspect of the present disclosure, an axis of one end of the image transmission optical fiber bundle that is fixed on the optical fiber bundle fixing structure is collinear with an axis of the image projection lens group.

In one aspect of the present disclosure, the support is detachably mounted on the table body, and the support comprises a support base and a mounting frame, the support base being detachably mounted on the table body, and the mounting frame being rotatably connected to the support base.

In one aspect of the present disclosure, a projection film is further comprised, the projection film being arranged on the top wall of the magnetic resonance chamber, and the image being projected to the projection film.

The present disclosure proposes an imaging apparatus for a magnetic resonance device. The imaging apparatus converts an image broadcast by an image broadcast apparatus into electrical signals by means of an optical/electrical conversion module, and then converts the electrical signals into an image by means of an electrical/optical conversion module, and by means of an image transmission assembly, transmits the image to a projection device which projects the image to the interior of a magnetic resonance chamber for a patient being examined to view, thereby calming the patient, ensuring the examination proceeds normally; a method of photoelectric separation is used thereby. A lens group and an image transmission optical fiber bundle are used to guide an image outside a magnet to inside the magnet, which can prevent electromagnetism from interfering therewith. Moreover, by means of the optical/electrical conversion module and the electrical/optical conversion module, the image is converted into electrical signals and then reconverted into an image, which is transmitted and projected to the interior of the magnetic resonance chamber to enhance projection quality of the image, with greater benefit for viewing by the patient.

The present disclosure proposes an imaging apparatus for a magnetic resonance device. In the imaging apparatus, an image transmission lens group is arranged inside an examination table so that the image transmission lens group replaces some of an image transmission optical fiber bundle to simultaneously realize the function of image size reduction and the function of image transmission, in order to replace a structure in which all of the image transmission is performed by the image transmission optical fiber bundle, so as to reduce the cost thereof and avoid movement of a table body being affected by the optical fiber bundle due to all image transmission being performed by the image transmission optical fiber bundle.

The present disclosure proposes an imaging apparatus for a magnetic resonance device, wherein a top wall of a magnetic resonance chamber is provided with a projection film, and an image is finally projected to the projection film to increase the resolution of the image. Moreover, when the distance between a patient's eyes and the top wall of the magnetic resonance chamber is small, it easily produces a feeling of discomfort, and so the present application uses the image projection lens group to perform projection, which is able to achieve an effect of pushing the image further away, to increase the viewing comfort of the patient, and the patient may directly view the image without needing to rely on devices such as glasses or a mirror for viewing.

The present disclosure proposes an imaging apparatus for a magnetic resonance device. In the imaging apparatus, an axis of one end of an image transmission optical fiber bundle that is fixed on the optical fiber bundle fixing structure is collinear with an axis of the image projection lens group to improve the projection quality thereof; at the same time, an angle of a support is adjustable, to facilitate use by patients in different positions.

### BRIEF DESCRIPTION OF THE DRAWINGS

To explain the technical solutions in aspects of the present disclosure more clearly, there follows a simple description of the accompanying drawings that need to be used in describing the aspects. Obviously, the drawings in the description below are merely some aspects of the present disclosure, and a person skilled in the art could obtain other drawings based on these drawings without expending any inventive effort.

FIG. 1 is a structural block diagram of an imaging apparatus in an aspect of the present disclosure.

FIG. 2 is a structural schematic diagram of an installation of the imaging apparatus and a magnetic resonance device in an aspect of the present disclosure.

### KEY TO LABELS

100, magnetic resonance device; 110, lifting assembly; 120, examination table; 130, scanning mechanism; 1021, base; 1022, table body; 101, magnetic resonance chamber; 10, image broadcast apparatus; 11, optical fiber; 20, optical/electrical conversion module; 30, electrical/optical conversion module; 40, image transmission assembly; 50, projection device; 12, electromagnetic shielding box; 41, image transmission lens group; 42, image transmission optical fiber bundle; 13, adjustment lens; 51, image projection lens group; 52, support; 1011, projection film.

### DETAILED DESCRIPTION

Aspects of the present disclosure are explained below by way of specific particular aspects; a person skilled in the art may easily understand other advantages and effects of the present disclosure from the content disclosed in this Description. The present disclosure may further be implemented or applied by way of other different particular aspects; various modifications or changes may also be made to various details in this Description, on the basis of different viewpoints and applications, without departing from the spirit of the present disclosure.

It must be explained that the illustrations provided in the present aspects merely schematically illustrate the basic concept of the present disclosure. The drawings only show relevant components in the present disclosure and are not drawn according to the numbers, shapes, and dimensions of components as actually implemented; the forms, numbers, and proportions of the various components when actually implemented may be arbitrarily changed, and the layout of the components may also be more complicated.

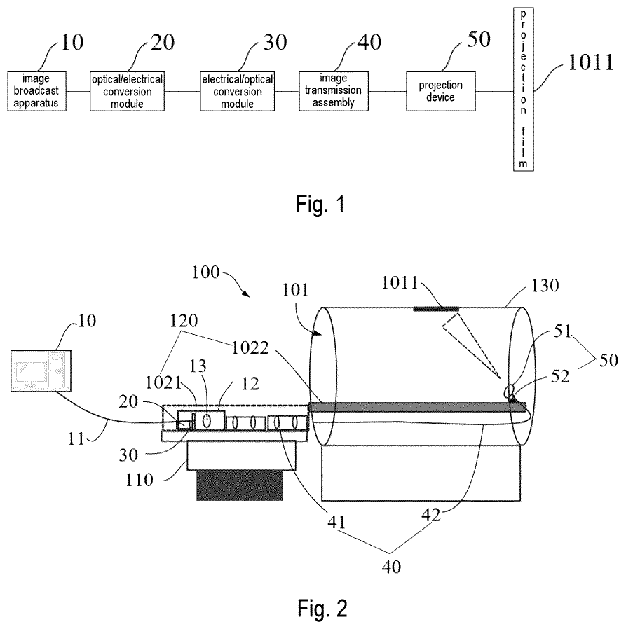

Referring to FIGS. 1 and 2, the present disclosure provides an imaging apparatus for a magnetic resonance device to mitigate the problem that it is difficult to add electronic indication signals inside magnets of existing nuclear magnetic devices, such that a patient must wait for a diagnosis to finish while lying inside a magnet, yet since the space in most existing magnetic resonance imaging devices is narrow and sealed, the patient easily becomes tense and anxious during the waiting process, thus affecting imaging quality. In the present aspect, the imaging apparatus is suitable for use in certain existing magnetic resonance devices 100; for example, the magnetic resonance device comprises a lifting assembly 110, an examination table 120, and a scanning mechanism 130, the examination table 120 comprising a base 1021 and a table body 1022, the base 1021 being mounted on the lifting assembly 110, the table body 1022 being slidably mounted on the base 1021, and a magnetic resonance chamber 101 also being formed in the scanning mechanism 130, and during examination, the table body 1022 sliding into the magnetic resonance chamber 101 and a scanning examination being performed. In the present aspect, the imaging apparatus comprises an image broadcast apparatus 10, an optical/electrical conversion module 20, an electrical/optical conversion module 30, an image transmission assembly 40, and a projection device 50; the image broadcast apparatus 10 is located in a first space, and is used for broadcasting images; the optical/electrical conversion module 20 is connected to the image broadcast apparatus 10, by means of an optical fiber 11 for example, and is used for converting an image broadcast by the image broadcast apparatus 10 into electrical signals; the electrical/optical conversion module 30 is connected to the optical/electrical conversion module 20, and is used for reconverting the electrical signals into the image; and the projection device 50 is connected to the electrical/optical conversion module 30 by means of the image transmission assembly 40, in order to project the image to a top wall of the magnetic resonance chamber 101. Furthermore, in the present aspect, the magnetic resonance device 100, the optical/electrical conversion module 20, the electrical/optical conversion module 30, and the projection device 50 are located in a second space, and the first space and the second space are electromagnetically shielded from each other.

Referring to FIGS. 1 and 2, in the present aspect, the image broadcast apparatus 10 is used for broadcasting images and may be configured as a device, such as a computer, which is able to broadcast images and video; for example, the images may be a timer, breathing guidance or a movie. In the present aspect, the image broadcast apparatus 10 may be provided with a signal conversion module that converts the image into optical signals, and the optical/electrical conversion module 20 is connected to the signal conversion module by means of an optical fiber, so as to convert the optical signals into electrical signals. In some other aspects, the image may also be converted into electrical signals by means of another apparatus. In the present aspect, the optical/electrical conversion module 20 is arranged inside the base 1021; for example, specifically, an electromagnetic shielding box 12 is provided in the base 1021, and the optical/electrical conversion module 20 is mounted inside the electromagnetic shielding box 12. In the present aspect, the electromagnetic shielding box 12 cannot contain elements such as iron, cobalt, nickel, etc., and, for example, may be configured as a structure made of red copper or aluminum to realize an electromagnetic shielding effect, thereby further preventing electromagnetism interfering therewith.

Referring to FIGS. 1 and 2, in the present aspect, the electrical/optical conversion module 30 is mounted inside the electromagnetic shielding box 12 and is connected to the optical/electrical conversion module 20 so as to convert the electrical signals into the image; for example, the electrical/optical conversion module 30 may be configured as a DLP optical engine, wherein the DLP optical engine is used for restoring the electrical signals to form the image broadcast by the image broadcast apparatus 10. It must be explained that, during examination of a patient, the optical/electrical conversion module 20 and the electrical/optical conversion module 30 are both located outside the magnetic resonance chamber 101, that is, remote from the magnetic resonance chamber 101, which to a certain degree can prevent electromagnetism of the magnetic resonance chamber 101 from interfering with these electronic devices, to ensure the normal operation thereof; moreover, both of the modules are located inside the electromagnetic shielding box 12 to further prevent electromagnetism from interfering therewith. It must be explained that, in the present application, by means of the optical/electrical conversion module 20 and the electrical/optical conversion module 30, the image is converted into electrical signals and then reconverted into an image that is transmitted and projected to the interior of the magnetic resonance chamber; compared to direct projection by a projector in a conventional structure, the present application may enhance projection quality of the image, for example by increasing the brightness of the image, with greater benefit for viewing by a patient.

Referring to FIGS. 1 and 2, in the present aspect, the projection device 50 is connected to the electrical/optical conversion module 30 by means of the image transmission assembly 40; for example, the projection device 50 is connected to the DLP optical engine by means of the image transmission assembly 40, so that, by means of the image transmission assembly 40, the image restored by the DLP optical engine is transmitted to the projection device 50, and, by means of the projection device 50, projected onto the top wall of the magnetic resonance chamber 101.

Referring to FIGS. 1 and 2, in the present aspect, the image transmission assembly 40 at least comprises an image transmission lens group 41 and/or an image transmission optical fiber bundle 42, and the image is transmitted to the projection device 50 by means of the image transmission lens group 41 and/or the image transmission optical fiber bundle 42. For example, the image transmission assembly 40 comprises the image transmission lens group 41 and the image transmission optical fiber bundle 42; two ends of the image transmission optical fiber bundle 42 are respectively connected to the image transmission lens group 41 and the projection device 50; in the image transmission process, after projection and scaling multiple times via the image transmission lens group 41, a real image that is reduced in size is finally projected to a terminal end of the image transmission optical fiber bundle 42 that is connected to the image transmission lens group 41, and, by means of the image transmission optical fiber bundle 42, the image is transmitted to an end of the image transmission optical fiber bundle 42 that is remote from the image transmission lens group 41, and finally, the projection device 50 that is connected to the image transmission optical fiber bundle 42 projects the image to the top wall of the magnetic resonance chamber 101; that is, the image transmission lens group 41 at least replaces some of the image transmission optical fiber bundle 42, and the image transmission lens group 41 simultaneously realizes the function of image size reduction and the function of image transmission, in order to replace a structure in which all of the image transmission is performed by the image transmission optical fiber bundle, so as to reduce the cost thereof, and avoid movement of the table body being affected by the optical fiber bundle due to all image transmission being performed by the image transmission optical fiber bundle. It must be explained that the image transmission lens group 41 may be arranged in the base 1021 and, of course, may also be arranged on the table body 1022 to at least replace some of the image transmission optical fiber bundle; of course, the image transmission lens group 41 may be partially arranged in a gap between a bottom wall of the magnetic resonance chamber 101 and the table body 1022. Of course, in some other aspects, the image transmission assembly 40 may further only be provided with the image transmission lens group 41 or the image transmission optical fiber bundle 42 to perform image transmission.

Referring to FIGS. 1 and 2, in the present aspect, an adjustment lens 13 is provided between the DLP optical engine and the image transmission lens group 20; the adjustment lens 13 is used for adjusting the position and size of the image so that the image is better projected into the image transmission lens group 20. In some other aspects, the adjustment lens 13 may slide between the DLP optical engine and the image transmission lens group 20 to cause the adjustment lens to adapt to different image transmissions.

Referring to FIGS. 1 and 2, in the present aspect, the projection device 50 is arranged on one end of the table body 1022 that is near the magnetic resonance chamber 101; for example, the projection device 50 comprises an image projection lens group 51 and a support 52, the support 52 is mounted on one end of the table body 1022 that is near the magnetic resonance chamber 101, the image projection lens group 51 is fixedly mounted on the support 52, one end of the image transmission optical fiber bundle 42 that is remote from the image transmission lens group 41 passing through the bottom of the table body 1022, and being connected to the image projection lens group 51, so as to transmit the image to the image projection lens group 51 which projects the image into the magnetic resonance chamber 101. It must be explained that when the distance between a patient's eyes and the top wall of the magnetic resonance chamber 101 is small, it easily produces a feeling of discomfort, and so the present application uses the image projection lens group 51 to perform projection, which is able to achieve an effect of pushing the image further away, to increase the viewing comfort of the patient.

Referring to FIGS. 1 and 2, in the present aspect, the top wall is provided with a projection film 1011, and the image is projected to the projection film 1011; the projection film 1011 may be a transparent projection film, for example, the image projection lens group 51 projects the image onto the top wall of the interior of the magnetic resonance chamber 101, to increase the resolution of the image, such that the patient may directly view the image without needing to rely on devices such as glasses or a mirror for viewing.

Referring to FIGS. 1 and 2, in the present aspect, the support 52 is detachably mounted on the table body 1022 to facilitate replacement or maintenance of the image projection lens group 51 according to requirements; the support 52 is mounted at one end of the table body 1022 that is near the magnetic resonance chamber 101, and the image projection lens group 51 is fixedly mounted on the support 52; the support 52 may move together with the table body 1022 to avoid frequently adjusting the position thereof. It must be explained that, in the present application, one end of the table body 1022 that is near or remote from the magnetic resonance chamber 101 refers to an end part of the table body 102 that is near/remote from the magnetic resonance chamber 101 before the table body has moved inside the magnetic resonance chamber 101. In the present aspect, the support 52 comprises a support base and a mounting frame, the support base being detachably connected to the table body 1022, the mounting frame is rotatably connected to the support base, and the image projection lens group 51 is fixedly mounted on the mounting frame, to facilitate adjustment of an angle of the image projection lens group 51 according to requirements, facilitating use by patients in different positions.

Referring to FIGS. 1 and 2, in the present aspect, the support 52 is further provided with an optical fiber bundle fixing structure, one end of the image transmission optical fiber bundle 42 that is remote from the image transmission lens group 41 being fixed on the optical fiber bundle fixing structure, to cause one end of the image transmission optical fiber bundle 42 that is near the image projection lens group 51 to always remain fixed relative to the image projection lens group 51, thereby avoiding image projection being affected due to movement of the table body. In the present aspect, an axis of one end of the image transmission optical fiber bundle 42 that is fixed on the optical fiber bundle fixing structure is collinear with an axis of the image projection lens group 51 to ensure projection quality thereof. In some other aspects, when the image transmission lens group 41 is fixedly mounted in the base 1021, the length of the image transmission optical fiber bundle 42 is greater than the distance between one end of the table body 1022 that is provided with the image projection lens group 51, when the table body 1022 is located inside the magnetic resonance chamber 101, and the image transmission lens group 41, to avoid the image transmission optical fiber bundle 42 being excessively pulled, and consequently damaged, when the table body moves, and furthermore, an electromagnetic shielding layer is wrapped on the outside of the image transmission optical fiber bundle 42, to prevent electromagnetic interference, improving the projection quality thereof. It must be explained that, in the present aspect, the optical fiber bundle fixing structure and the mounting frame are an integral structure, such that during angle adjustment thereof, the image transmission optical fiber bundle 42 and the image projection lens group 51 are able to rotate synchronously, which can avoid the image transmission optical fiber bundle 42 and the image projection lens group 51 being displaced relative to each other after angle adjustment and affecting image projection.

The present disclosure proposes an imaging apparatus for a magnetic resonance device. The imaging apparatus converts an image broadcast by an image broadcast apparatus into electrical signals by means of an optical/electrical conversion module, and then converts the electrical signals into an image by means of an electrical/optical conversion module, and by means of an image transmission assembly, transmits the image to a projection device which projects the image to the interior of a magnetic resonance chamber for a patient being examined to view, thereby calming the patient, ensuring the examination proceeds normally; a method of photoelectric separation is used thereby. A lens group and an image transmission optical fiber bundle are used to guide an image outside a magnet to inside the magnet, which can prevent electromagnetism from interfering therewith. Moreover, by means of the optical/electrical conversion module and the electrical/optical conversion module, the image is converted into electrical signals and then reconverted into an image, which is transmitted and projected to the interior of the magnetic resonance chamber to enhance projection quality of the image, with greater benefit for viewing by the patient.

The present disclosure proposes an imaging apparatus for a magnetic resonance device. In the imaging apparatus, an image transmission lens group is arranged inside an examination table so that the image transmission lens group replaces some of an image transmission optical fiber bundle to simultaneously realize the function of image size reduction and the function of image transmission, in order to replace a structure in which all of the image transmission is performed by the image transmission optical fiber bundle, so as to reduce the cost thereof and avoid movement of a table body being affected by the optical fiber bundle due to all image transmission being performed by the image transmission optical fiber bundle.

The present disclosure proposes an imaging apparatus for a magnetic resonance device, wherein a top wall of a magnetic resonance chamber is provided with a projection film, and an image is finally projected to the projection film to increase the resolution of the image. Moreover, when the distance between a patient's eyes and the top wall of the magnetic resonance chamber is small, it easily produces a feeling of discomfort, and so the present application uses the image projection lens group to perform projection, which is able to achieve an effect of pushing the image further away, to increase the viewing comfort of the patient, and the patient may directly view the image without needing to rely on devices such as glasses or a mirror for viewing.

The present disclosure proposes an imaging apparatus for a magnetic resonance device. In the imaging apparatus, an axis of one end of an image transmission optical fiber bundle that is fixed on the optical fiber bundle fixing structure is collinear with an axis of the image projection lens group to improve the projection quality thereof; at the same time, an angle of a support is adjustable, to facilitate use by patients in different positions.

It should be understood that the term “one aspect,” “aspect,” or “particular aspect” mentioned throughout the Description means that a specific feature, structure, or characteristic described in conjunction with an aspect is included in at least one aspect of the present disclosure, and not necessarily in all aspects. Accordingly, various expressions of the phrase “in one aspect,” “in an aspect,” or “in a particular aspect” used in different places throughout the Description do not necessarily refer to the same aspect. In addition, a specific feature, structure, or characteristic of any particular aspect of the present disclosure may be combined with one or more other aspects in any appropriate way. It should be understood that other variations and modifications of the aspects of the disclosure described and illustrated herein may be based on the teaching herein and are considered a part of the spirit and scope of the present disclosure.

It should further be understood that one or more elements shown in the drawings may also be implemented in a more separate or more integrated manner, or even be removed due to inoperability in certain situations, or may be provided due to utility according to specific applications.

The above description is merely a description of preferred aspects of the present application and the technical principles employed. A person skilled in the art will understand that the scope of the present application is not limited to technical solutions formed by specific combinations of the technical features described above but also covers other technical solutions formed by any combination of the above technical features or equivalent features without departing from the concept of the present disclosure, such as technical solutions formed by interchanging the above features and technical features having a similar function which the present application discloses (but without limitation).

Besides the technical features of the Description, the remaining technical features are technology known to a person skilled in the art, and to highlight the innovative characteristics of the present disclosure, the remaining technical features are not unnecessarily detailed here.

1. An imaging apparatus for a magnetic resonance device having an examination table and a magnetic resonance chamber, the imaging apparatus comprising:
an image broadcast apparatus located in a first space;
an optical/electrical conversion module, which is connected to the image broadcast apparatus using an optical fiber, and is configured to convert an image broadcast by the image broadcast apparatus into electrical signals;
an electrical/optical conversion module, which is connected to the optical/electrical conversion module and is configured to convert the electrical signals into the image; and
a projection device, which is connected to the electrical/optical conversion module using an image transmission assembly, and is configured to project the image to a top wall of the magnetic resonance chamber, wherein the image transmission assembly comprises an image transmission lens group and/or an image transmission optical fiber bundle,
wherein the magnetic resonance device, the optical/electrical conversion module, the electrical/optical conversion module, and the projection device are located in a second space, and the first space and the second space are electromagnetically shielded from each other.

2. The imaging apparatus as claimed in claim 1, wherein the electrical/optical conversion module is a DLP optical engine.

3. The imaging apparatus as claimed in claim 1, wherein the projection device is arranged at an end of a table body of the examination table that is near the magnetic resonance chamber.

4. The imaging apparatus as claimed in claim 1, further comprising:
an electromagnetic shielding box, which is mounted inside a base of the examination table, wherein the optical/electrical conversion module and the electrical/optical conversion module are arranged inside the electromagnetic shielding box.

5. The imaging apparatus as claimed in claim 1, wherein the image transmission lens group is mounted inside a base of the examination table or on a table body of the examination table.

6. The imaging apparatus as claimed in claim 1, wherein the image transmission assembly comprises an image transmission lens group and an image transmission optical fiber bundle, and two ends of the image transmission optical fiber bundle are respectively connected to the image transmission lens group and the projection device.

7. The imaging apparatus as claimed in claim 6, wherein the projection device comprises a support and an image projection lens group, the support being mounted at an end of a table body of the examination table that is near the magnetic resonance chamber, and the image projection lens group is fixed on the support.

8. The imaging apparatus as claimed in claim 7, wherein the support comprises an optical fiber bundle fixing structure, and an end of the image transmission optical fiber bundle that is remote from the image transmission lens group is fixed on the optical fiber bundle fixing structure.

9. The imaging apparatus as claimed in claim 8, wherein an axis of an end of the image transmission optical fiber bundle that is fixed on the optical fiber bundle fixing structure is collinear with an axis of the image projection lens group.

10. The imaging apparatus as claimed in claim 7, wherein the support is detachably mounted on the table body, and the support comprises a support base and a mounting frame, the support base being detachably mounted on the table body, and the mounting frame being rotatably connected to the support base.

11. The imaging apparatus as claimed in claim 1, further comprising:
a projection film arranged on a top wall of the magnetic resonance chamber, wherein the image is projectable to the projection film.
